(19) United States
(12) Patent Application Publication
(10) Pub. No.: US 2010/0099751 A1
(43) Pub. Date: Apr. 22, 2010

(54) Method of treating diabetes-related vascular complications

(76) Inventors: Al-Mulla Fahd, Al-Yarmouk (KW); Bitar Milad, Al-Yarmouk (KW)

Correspondence Address: LITMAN LAW OFFICES, LTD., POST OFFICE BOX 41200, SOUTH STATION, ARLINGTON, VA, 22204, US

(21) Appl. No.: 12/289,146

(22) Filed: Oct. 21, 2008

(51) Int. Cl. A61K 31/385; A61P 3/10

(52) U.S. Cl. 514/440

(57) ABSTRACT

A method of treating diabetes-related vascular complications is provided. It has been found that a heightened state of oxidative stress, either acting alone or in concert with augmented apoptotic and inflammatory processes, contributes to diabetes-related vascular dysfunction. The method of treating diabetes-related vascular complications includes the treatment of diabetic patients with alpha-lipoic acid (LA) in order to mitigate the negative impact of diabetes-related vascular dysfunctions upon vascular homeostasis. The treatment method includes the step of administering to the patient a therapeutically effective dosage of alpha-lipoic acid.

### BACKGROUND OF THE INVENTION

1. Field of the Invention

The present invention relates to the treatment of diabetes-related vascular complications. The treatment method includes the step of administering to the patient a therapeutically effective dosage of alpha-lipoic acid.

2. Description of the Related Art

Epidemiological and experimental evidence both indicate that diabetes is a major risk factor for the development of atherosclerosis and hypertension, and these clinical scenarios lead to aortic aneurysm, heart failure, myocardial infraction and stroke. It has been shown that the diabetic vascular system is associated with endothelial dysfunction and this phenomenon is considered to be a causal factor in the development of atherothrombotic disease, and as one of the earliest abnormalities that can be detected clinically in an individual predisposed to atherosclerosis and hypertension. However, the exact molecular mechanisms responsible for these changes in vascular phenotype in diabetes remain unknown. Further, treatment intended to reverse or delay diabetes-induced decline of vascular function has yet to be implemented.

Dysfunction of the endothelium in a number of vascular diseases, including diabetes, hypertension and atherosclerosis, is associated with reduced bioavailability of the signaling molecule nitric oxide, which has potent vasodilatory, anti-inflammatory and antiatherosclerotic properties. A large quantity of available evidence indicates that impaired endothelium-derived NO bioavailability is due, in part, to excess oxidative stress. Diseased blood vessels produce increased levels of reactive superoxide anion (O₂—) and hydrogen peroxide. Superoxide anion reacts with NO, yielding peroxynitrate, which has the potential of inducing protein modification, DNA damage, apoptosis and inflammation.

Oxidative stress in a physiological setting reflects an excessive bioavailability of ROS, which is the net result of an imbalance between production and destruction of ROS, with the latter being influenced by antioxidant defenses, including antioxidant enzyme (e.g., superoxide dismutase, glutathione peroxidase, and catalase) and chemical antioxidants (e.g., α-lipoic acid (LA) and vitamins). Excessive stress has been shown to promote apoptosis and elicits several inflammatory responses in endothelial cells, including the production of proinflamatory responses in endothelial cells, including the production of proinflammatory cytokines and chemokines TNF-α, IL-β, along with monocyte chemoattractive protein MCP-1, and an increased surface expression of the cellular adhesion molecules, E-selectin, vascular cell adhesion molecule 1 (VCAM-1) and intracellular adhesion molecule (ICAM-1). A large portion of the above parameters are altered as a function of diabetes.

α-lipoic acid (LA) is an endogenous short-chain fatty acid which occurs naturally in the human diet and is rapidly absorbed and converted intracellularly to dihydrolipoic acid via NAD(P)H-dependent enzymes. In addition to playing an important role as a cofactor for mitochondrial bioenergetic enzymes, LA and dihydrolipoic acid can scavenge ROS, regenerate other natural antioxidants, such as glutathione, vitamin C and vitamin E, chelate metals ions, and stimulate insulin signaling. LA further improves neurovascular and metabolic abnormalities and may further play a role in cardiovascular protection and as an anti-inflammatory agent. Additionally, it has been shown that LA ameliorates diabetes-related deficits in skeletal muscle glucose metabolism, protein oxidation, as well as the activation by insulin of the various steps of the insulin signaling pathway, including the enzymes AKT/PKB and phosphatidyl inositol 3-kinase.

Thus, a method of treating diabetes-related vascular complications solving the aforementioned problems is desired.

### SUMMARY OF THE INVENTION

It has been found that a heightened state of oxidative stress, either acting alone or in concert with augmented apoptotic and inflammatory processes, contributes to diabetes-related vascular dysfunction. The method of treating diabetes-related vascular complications includes the treatment of diabetic patients with alpha-lipoic acid (LA) (sometimes alternately written as α-lipoic acid) in order to mitigate the negative impact of diabetes-related vascular dysfunctions upon vascular homeostasis. The treatment method includes the step of administering to the patient a therapeutically effective dosage of alpha-lipoic acid.

In human patients, the effective dosage of alpha lipoic acid is preferably between approximately 100 and 300 mg., delivered daily. Although the alpha lipoic acid may be injected in solution, it is preferably delivered orally to the patient.

These and other features of the present invention will become readily apparent upon further review of the following specification and drawings.

### BRIEF DESCRIPTION OF THE DRAWINGS

FIG. 1 is a data plot illustrating relaxation in aortic vessels as a function of maximum norepinephrine-induced vasoconstriction.

FIG. 2 is a graph illustrating aortic superoxide production in a control sample, a diabetic sample, and in alpha lipoic acid-treated rats.

FIG. 3 illustrates ethidium bromide fluorescent photomicrographs of control, diabetic and alpha lipoic acid-treated diabetic rats.

FIG. 4 is a graph illustrating NAD(P)H-based O₂ production in aortic homogenates in a control sample, a diabetic sample, and in alpha lipoic acid-treated rats.

FIG. 5A is a graph illustrating gp 91^{phox} concentration in blood vessels in a control sample, a diabetic sample, and in alpha lipoic acid-treated rats.

FIG. 5B is a graph illustrating nox-1 concentration in blood vessels in a control sample, a diabetic sample, and in alpha lipoic acid-treated rats.

FIG. 6A is a graph illustrating aortic contents of protein-bound carbonyls in a control sample, a diabetic sample, and in alpha lipoic acid-treated rats.

FIG. 6B is a graph illustrating aortic contents of TBARS in a control sample, a diabetic sample, and in alpha lipoic acid-treated rats.

FIG. 7A is a graph illustrating DNA fragmentation in a control sample, a diabetic sample, and in alpha lipoic acid-treated rats.

FIG. 7B is a graph illustrating caspase 3/7 activity in aortic rat vessels in a control sample, a diabetic sample, and in alpha lipoic acid-treated rats.

FIG. 8A is a graph illustrating plasma levels in a control sample, a diabetic sample, and in alpha lipoic acid-treated rats.

FIG. 8B is a graph illustrating aortic mRNA expression of TNF-α in a control sample, a diabetic sample, and in alpha lipoic acid-treated rats.

FIG. 9A is a graph illustrating superoxide generation as a function of TNF-α.

FIG. 9B is a graph illustrating relative DNA fragmentation as a function of TNF-α.

FIG. 9C is a graph illustrating acetylcholine induced vasorelaxation as a function of TNF-α.

FIG. 10A illustrates western blot analyses of Nf-κβ protein expression in aortic tissues of CTL GK and GK+LA rats.

FIG. 10B illustrates averaged densitometric data for a diabetic sample and a sample treated with alpha lipoic acid expressed as a percentage of change over CTL values.

FIG. 11A is a graph illustrating mRNA expression of IL-6 in a control sample, a diabetic sample, and in alpha lipoic acid-treated rats.

FIG. 11B is a graph illustrating CMA mRNA expression in a control sample, a diabetic sample, and in alpha lipoic acid-treated rats.

Similar reference characters denote corresponding features consistently throughout the attached drawings.

### DETAILED DESCRIPTION OF THE PREFERRED EMBODIMENT

The present invention is directed towards a method of treating diabetes-related vascular complications. It has been found that a heightened state of oxidative stress, either acting alone or in concert with augmented apoptotic and inflammatory processes, contributes to diabetes-related vascular dysfunction. The present invention is directed towards the treatment of diabetic patients with α-lipoic acid (LA) in order to mitigate the negative impact of the above dysfunction upon vascular homeostasis. The treatment method includes the step of administering to the patient a therapeutically effective dosage of alpha-lipoic acid.

It has been further found that diabetic aortic tissue exhibits a decline in acetylcholine-induced relaxation and a heightened state of oxidative stress (as exemplified by an increase in NAD(P)H oxidase activity and expression), elevation in the levels of protein-bound carbonyle and thiobartbituric acid reactive substance, along with an enhancement in the rate of superoxide production, aortic DNA fragmentation rate and caspase 3/7 activity. Further, sensitive indicators of the rate of apoptotic cell death are augmented as a function of diabetes. Similarly, an upregulation in vascular inflammatory markers, including TNFα, IL-6, intracellular adhesion molecule 1 and monocyte chemoattractant protein-1 (MCAP-1), is evident in this disease state.

Additionally, an assessment of nuclear factor kappa β activity (NF-κβ) reveals a marked accumulation of this transcriptional factor in aortic nuclear extracts of diabetic rats. At least a portion of the above abnormalities may be reversed following a chronic treatment of the diabetic patient with LA.

In aortic tissue of control animals, it has been found that TNFα elicits endothelial dysfunction, augmented state of oxidative stress, increased apoptosis and pro-inflammatory gene expression, mimicking in many respects the clinical features of diabetic vessels. Thus, it can be concluded that LA exerts vasculoprotective effects, possibly via mechanisms involving the down regulation of the TNFα/NFκβ signaling pathway. It has further been concluded that α-lipoic acid mitigates the negative impact of the aforementioned phenomena upon diabetic vascular homeostasis through the PI3K/Akt signaling pathway.

In the below, a study has been performed to examine the reversing or delaying of certain pathphysiological features of diabetes-mediated endothelial dysfunction in the therapeutic context of chronic intraperitoneal administration of LA to Goto-Kakasaki (GK) rats, a generic animal model of non-obese type II diabetes. Though the below experimental data and descriptions are based upon rat physiologies, extrapolated for human usage, the proper dosage in humans is preferably between approximately 100 mg. and 300 mg., taken daily. Though alpha lipoic acid may be injected in solution, the patient preferably receives the dosage orally.

In the experiments, with regard to animals and drug treatment, animal studies were performed in accordance with the National Institutes of Health Guidance for the care and use of laboratory animals. Type II diabetic GK rats were produced by selective inbreeding of glucose-intolerant Wistar rats. All offspring of GK animals are similarly affected by mild hyperglycemia within the first two weeks of birth. Weight-matched male Wistar rats served as a control. Three groups of animals were studied: vehicle-treated Wistar rats (n=8), vehicle-treated GK rats (n=10) and α-LA-treated GK rats (n=12). α-LA at a concentration of 50 mg./kg., i.p. (Calbiochem La Jolla Calif.) dissolved in tris-base and adjusted to a pH of 7.4 was injected daily for a duration of four weeks. All rats were maintained under a 12-hour light-dark cycle and had free access to water and a standard rodent's diet.

In the experiments, with regard to the determination of endothelium dependent relaxation (EDR) in the aorta, EDR in response to various concentrations of Ach (10⁻⁹ to 10⁻⁶ mol/l) was assessed in norepinephrine (10⁻⁷ mol/l) preconstructed rat aortic rings using an organ chamber bath. The effects of the NAD(P)H oxidase inhibitor apocynin (3×10⁻⁴ mol/l) and the O₂— scavenger Tiron (10 mmol/l) on Ach-induced responses of diabetic arteries were also considered.

In the study, with regard to measurement of vascular superoxide anion formation, O₂— concentration in aortic tissue was determined using a lucigenin enhanced chemiluminescence method and the resulting data were further confirmed by a cytochrome c-based technique. Segments of the thoracic aorta were placed into 2 ml Krebs-Henseleit buffer (KHB, pH 7.4), and prewarmed to 37° C. for one hour. Immediately before measurement, rings were transferred to scintillation vials containing KHB with 5 μmol/L lucigenin and the O₂— generated chemiluminescence was recorded for five minutes with a scintillation counter. The amount of O₂— produced was quantified using a standard curve of O₂— generation by xanthine/xanthine oxidase and the data are expressed as nmol per min, per mg, of wet weight. In some experiments, vessels were denuded of endothelium by gentle rubbing of the luminal surface, whereas in others, N^{ω}-nitro-L-arginine methyl ester (L-NAME) 0.1 mM, diphenylene iodonium 0.1 mM, or apocynin 3 mM were added 60 min before determining O₂— generation.

Dihydroethidium (DHE), an oxidative fluorescent dye, was used to localize superoxide production in situ. DHE is oxidized on reaction with superoxide to ethidium bromide, which binds to DNA in the nucleus and fluoresces. Arteries were embedded in OCT medium, frozen and cryosectioned. Vascular sections were incubated with DHE at a concentration (10⁻⁶ mol/l) at 37° C. for 30 minutes. DHE images from serial sections were obtained using a Zeiss Axioplan 2000 fluorescence microscope.

Superoxide production was also determined using the superoxide dismutase (SOD)-inhibitable cytochrome c assay. Three to four aortic ring segments (2 mm.) were placed in a buffer containing (in mM) NaCl 145, KCl 4.86, Na₂HPO₄ 5.7, CaCl₂ 0.54, MgSO⁴ 1.22, glucose 5.5, deferoxamine mesylate 0.1, and 1 U/ml catalase. Cytochrome c (50 μM) was added and the reaction mixture was incubated at 37° C. for 60 min. with or without SOD (200 U/ml). Cytochrome c reduction was measured by reading absorbance at 550 nm. O₂— formation in nmol/mg protein was calculated from the difference between absorbance with or without SOD, and the extinction coefficient for change of ferricytochrome c to ferrocytochrome c, i.e., 21 mM/cm⁻¹.

Determination of NAD(P)H oxidase activity in the aorta was determined based on superoxide induced lucigenin photoemission. Enzyme assays were carried out in a final volume of 1 ml. containing (in mM) 50 phosphate buffer; pH 7.0, 1 EGTA, 150 sucrose, 0.5 lucigenin, 0.1 NAD(P)H and tissue homogenate. Enzyme reactions were initiated with the addition of lucigenin. Photoemission, expressed in terms of relative light units (RLU), was measured every 5 min. using a luminometer. All assays were carried out in the dark at room temperature. NADPH oxidase-derived O₂— was confirmed using the flavo protein inhibitor diphenyleneiodinium, which reduced production of O₂— by >95% in the homogenate.

NADPH oxidases, the primary catalysts for the generation of reactive oxygen species (ROS), in terms of activities and levels of mRNA expression (e.g., nox 1, gp91 phox subunits) together with the established indices of oxidative stress (e.g. protein-bound carbonyls, thiobarbituric acid reactive substance), were elevated in aortic tissue of the GK diabetic rats. An assessment of the dynamic status of nuclear factor kappa β (NF-κβ) in aortic tissues revealed that the diabetic state promotes its nuclear localization with a concomitant increase in NFkB-DNA binding activity. A substantial decrease in vascular activity of PI3K and its down stream target p-Akt was evident as a function of diabetes. Most of the aforementioned vascular abnormalities in diabetic animals were ameliorated following chronic LA therapy. It should be noted that wortmannin, a known inhibitor of PI3K, given chronically to GK rats, negated the anti-inflammatory and anti-apoptotic actions of LA. In aortic tissue of control animals, TNFα elicited endothelial dysfunction, augmented state of oxidative stress, increased apoptosis and pro-inflammatory gene expression, mimicking in many respects the clinical features of diabetic vessels. Thus, it can be concluded that LA exerts vasculoprotective effect in diabetic animals by activating the PI3K/Akt signaling pathway.

Further, with regard to quantitative real-time polymerase chain reactions (PCR) in the study, total RNA from the arterial samples was isolated using TRIZOL® reagent, and RNA integrity was verified by agarose gel electrophorosis and quantified by spectrophotometry. Reverse transcription reaction of total RNA (5 μg) was performed using a superscript 111 first-strand synthesis system. Quantitative real-time PCR was performed using fast SYBR Green QPCR. Specific primers were as follows: TNF-αsense, 5⁻-TCG TAG CAA ACC ACC AAG-3⁻ and antisense, CTG ACG GTG TGG GTG A-3⁻-; gp 91^{phox} sense, 5⁻-GGA TGA ATC TCA GGC CAA-3⁻ and antisense-TTA GCC AAG GCT TCG G-3⁻; nox 1 sense, 5⁻-TGA. ATC TTG CTG GTT GAC ACT TGC-3⁻ and antisense, 5⁻GAG GGA CAG GTG GGA GGG AAG-3⁻; beta-Actin sense, 5⁻GAA GTG TGA CGT TGA CAT-3⁻ and antisense, 5⁻-ACA TCT GCT GGA AGG TG-3⁻.

The housekeeping gene beta-actin was used for internal normalization. Fidelity of the PCR reaction was determined by melting temperature analysis. PCR efficiency for each primer pair was determined by quantitating amplification with increasing concentration of template cDNA. A non-template control served as negative control to exclude the formation of primer dimmers or any other non-specific PCR products. RNA expression of target genes was calculated based on the real-time PCR efficiency E and the threshold crossing point (CP) and is expressed relative to the reference gene beta-actin.

With regard to lipid peroxidation, aortic tissues were homogenized in ice-cold tris-hydrochloric acid/buffer (pH 7.4) and butylated hydroxytoluene (BHT). Homogenates were centrifuged at 3,000×g at 4° C. for 10 min. An aliquot of the supernatant was combined with N-methyl-2-phenylindol (10.3) mmol/l in acetonitrile and methanol in the presence of methane sulfonic acid and BHT and the amount of malondialdehyde and 4-hydroxy-2-nonenal was assessed.

With regard to the assessment of apoptotic cell death using enzyme-linked immuno-absorbent-based assay, aortic tissues derived from control, GK and LA-treated GK rats were lysed and cytoplasmic histone-associated DNA fragments, indicating apoptotic cell death were determined by the Cell Death Eliza® plus kit. Data are reported as arbitrary optical density units normalized to protein concentration.

For detection of caspase 3-like activity, protein was isolated and caspase activity was detected in resulting supernatant using an APO-ONE homogenous caspase 3/7 assay (Promega). With regard to subcellular fractionation and western blotting, aortic tissue nuclear extracts were prepared and protein (40 μg) was loaded in each well of 12.1 Tris HCl polyacrylamide gel. Seperated polypeptide was transferred to nitrocellulose membrane IBio-Rad) and probed with anti NF-κβ at a 1:2,000 liter. Chemiluminescent detection was performed by an ECL Western Blotting Detection Kit®.

Plasma TNF-α levels from various experimental groups were determined using a rat TNF-α Eliza kit and tissue protein content was determined using bovine serum albumin as a standard.

Data were normalized with respect to control mean values and expressed as means±SEM. Statistical analyses of data were conducted using the student t-test or by two-way analysis of variance followed by the Tukey post hoc test, as appropriate. Statistical significance was assumed at P<0.05.

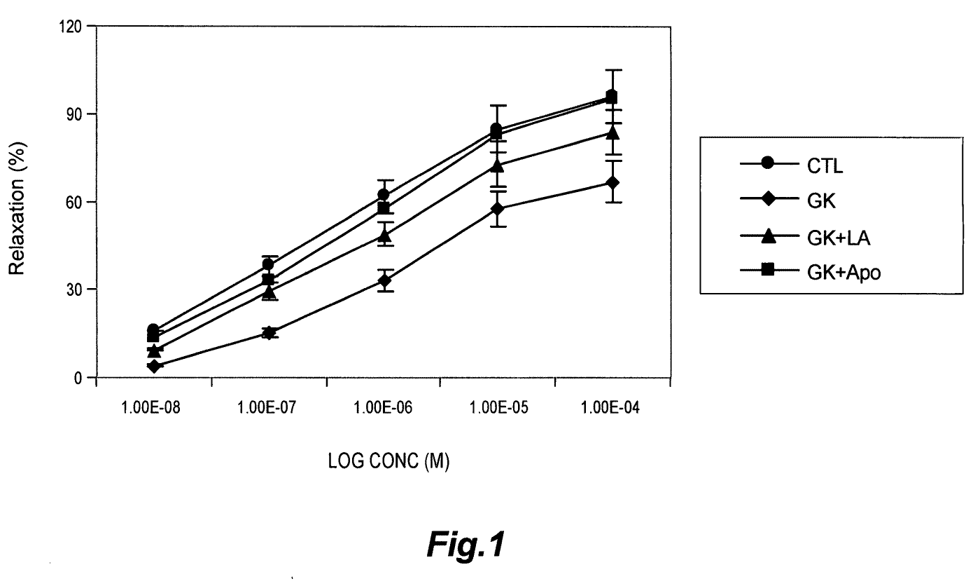

The experiments conducted in association with the present inventive method have shown that α-lipoic acid (LA) prevents oxidative stress-induced impairment in endothelial vasodilatory function during diabetes. A decline in acetylcholine (Ach)-induced relaxation of rat aorta was confirmed in GK diabetic rats, a phenomenon appearing to be ameliorated with LA (shown in FIG. 1). This beneficial effect of LA was not evident two weeks after its discontinuation. Both apocyanin and tiron improved Ach-induced relaxation in diabetic arteries, consistent with the concept that upregulation of NAD(P)H oxidase activity as being responsible, at least in part, for diabetes-induced endothelial dysfunction.

FIG. 1 illustrates relaxation to Acetylcholine(Ach) in aortic vessels of control (CTL), diabetic (GK), and LA-treated diabetic rats (GK+LA). Aortic segments of CTL, GK and GK+LA rats were isolated and their functional performance was assessed within an organ chamber. The graph of FIG. 1 shows force of contraction expressed as percentage of maximum norepinephrine-induced vasoconstriction. Data are expressed as means±SEM of at least 7 animals/group.

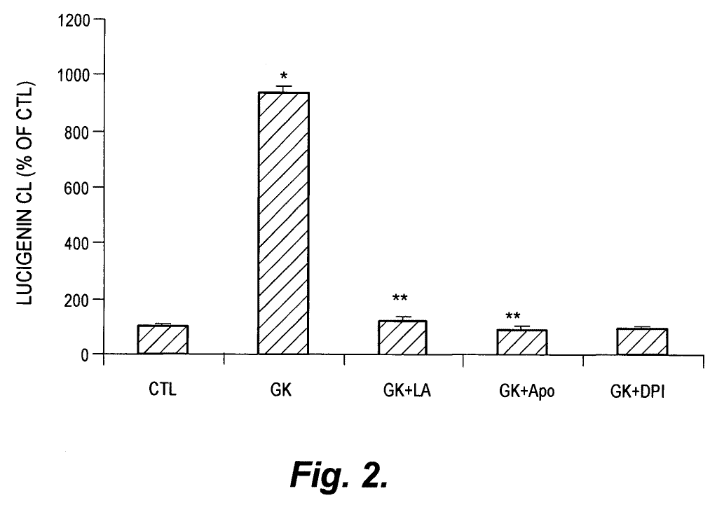

Lucigenin chemiluminescence measurement revealed that the aorta of GK diabetic rats exhibited a marked increase in O₂— production, which was inhibited by apocynin and diphenyleneiodionium, as shown in FIG. 2. FIG. 2 illustrates LA suppression of diabetes-mediated increases in aortic superoxide production in control (CTL), diabetic (GK), and LA-treated diabetic rats (GK+LA). Superoxide production was measured using a lucigenin chemiluminescence-based technique. Data are expressed as means±SEM of at least 7 animals/group. The “*” in FIG. 2 denotes significantly different values from corresponding CTL values at P<0.05. The “**” in FIG. 2 denotes significantly different values from corresponding vehicle treated diabetic values at P<0.05.

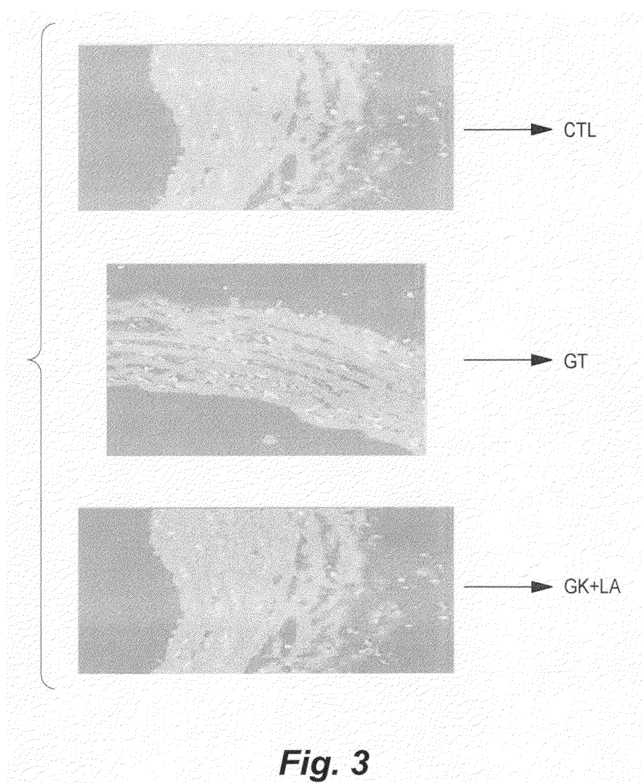

It should be noted that LA action on diabetic aortic O₂— generation mimics those produced by apocynin and diphenyleneiodonium. Immunohistochemistry-based techniques revealed that diabetic vessels exhibited a marked increase in the number of ethidium bromide (EB) positive nuclei, both in the endothelium (arrows) and media (smooth muscle cells) when compared to non-diabetic controls, as shown in FIG. 3. Further, nuclear EB fluorescence was significantly reduced in LA-treated diabetic rats. FIG. 3 illustrates ethidium bromide (EB) fluorescent photomicrographs of control (CTL), diabetic (GK), and LA-treated diabetic rats (GK+LA). The photomicrographs show representative images of EB stained nuclei in aortic vessels of CTL, GK and GK+LA rats.

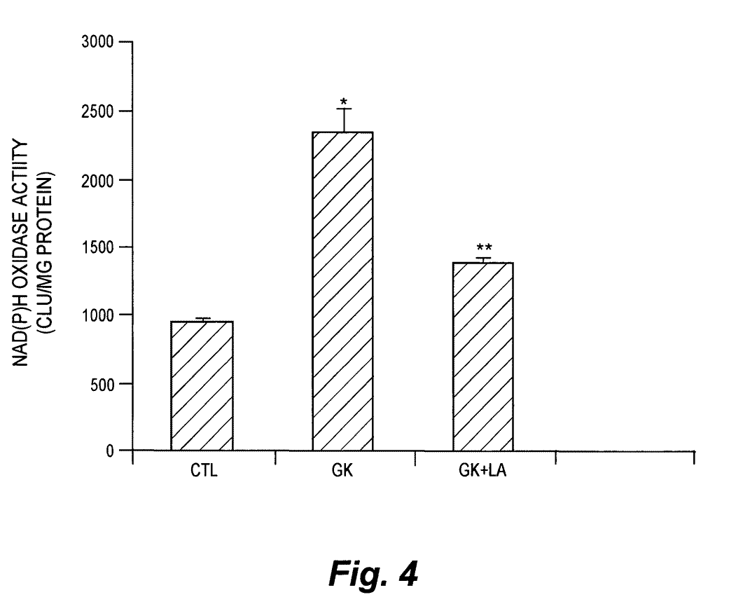

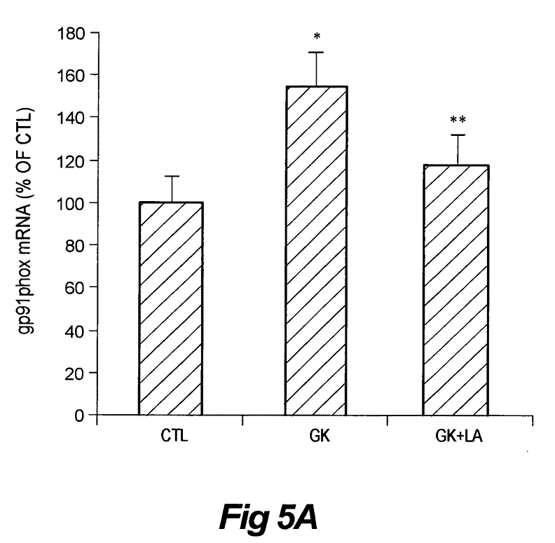

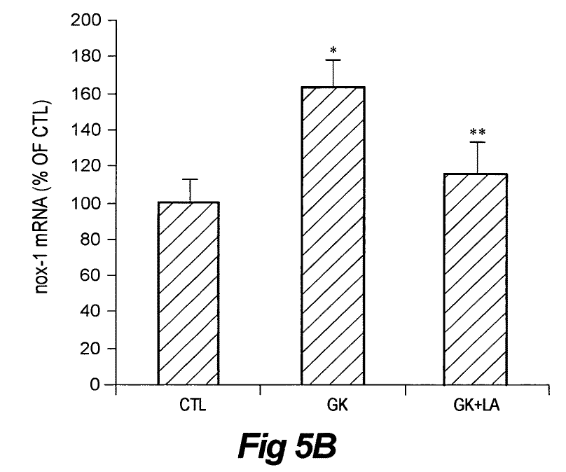

Further experimentation assessed NAD(P)H oxidase in terms of activity and gene expression in control, diabetic and a-LA treated diabetic rats. The data revealed an enhancement in NAD(P)H oxidase driven O₂— generation in homogenates of diabetic aorta, which was significantly attenuated following the institution of LA therapy, as shown in FIG. 4. LA treatment also tended to reduce the rate of gene expression of pg 9^{phox}, and nox-1 subunits, illustrated in FIGS. 5A and 5B. In FIG. 4, NAD(P)H-based O₂ production in aortic homogenates is shown of control (CTL), diabetic, and (GK) LA-treated diabetic rats (GK+LA). Lucigenin chemiluminescence-based techniques were used to measure the rate of aortic O₂ generation. Data are expressed as means±SEM of at least 7 animals/group. In FIG. 5A, expression of gp 91^{phox} is shown, and in FIG. 5B, expression of nox-1 is shown, both in vessels of control (CTL), diabetic (GK) and LA-treated diabetic rats (GK+LA). Analysis of mRNA expression was performed using RT-PCR based techniques.

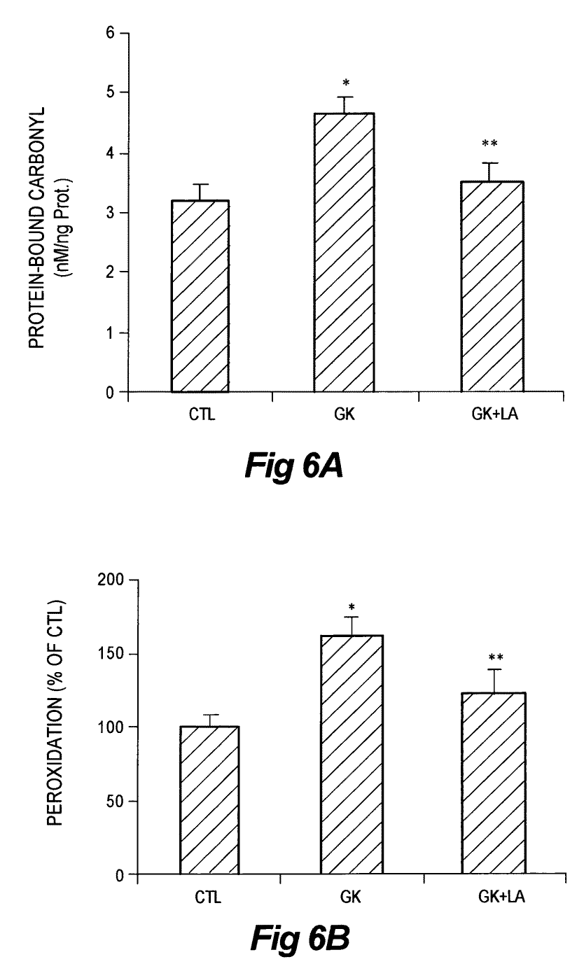

Overall, the above data are consistent with the concept that the diabetic aorta exhibits a heightened state of oxidative stress. The consequences of this phenomenon upon biological molecules including lipids and proteins were then determined. As can be seen in FIGS. 6A and 6B, the levels of both protein-bound carbonyls and the thiobarbituric acid reactive substances (an indicator of lipid peroxidation) were elevated in diabetic aorta by 45% and 60%, respectively. LA treatment partially reversed the oxidative stress-mediated damage to the lipid and protein molecules during diabetes. FIG. 6A illustrates aortic contents of protein-bound carbonyls in control (CTL), diabetic (GK) and LA-treated diabetic rats (GK+LA), and FIG. 6B illustrates aortic contents of TBARS in control (CTL), diabetic (GK) and LA-treated diabetic rats (GK+LA). Markers of the oxidative stress including protein-bound carbonyls and thiobarbituric acid reactive substances (TBARS) were measured in aortic homogenates. Data are expressed as means±SEM of at least 7 animals/group.

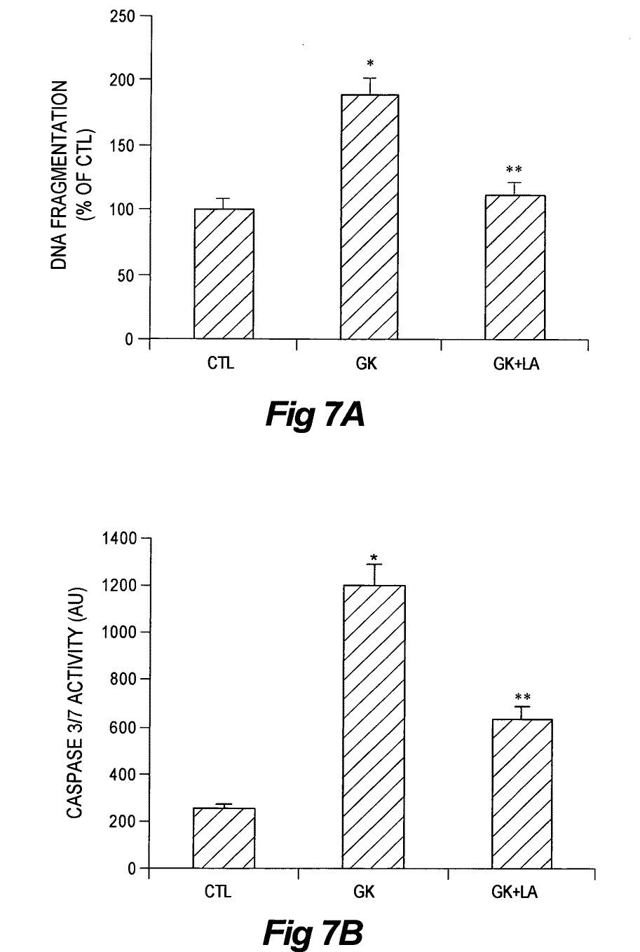

The experiments also showed that LA negates diabetes-induced apoptotic cell death. Cytotoxic DNA fragmentation and caspase activities are sensitive indicators of endothelial cell death in blood vessels. Thus, the levels of these parameters were measured in the aorta of various experimental groups including control (CTL), diabetic (GK) and LA-treated diabetic rats (GK+LA). As shown in FIG. 7A, the data reveals that the rate of DNA fragmentation in diabetic specimens was elevated by 75% over corresponding control values. In FIG. 7A, it is shown that LA negates diabetes-dependent increases in DNA fragmention at caspase 3/7 activity in aortic rat vessels. Markers of apoptotic cell death, including cytoplasmic histone-associated cell death and caspase 3/7 activity (shown in FIG. 7B) were assessed in aortic homogenates. Data are expressed as means±SEM of at least 7 animals/group. Chronic LA treatment significantly reduces DNA fragmentation rate by 42% and caspase 3/7 by 48% in diabetic arteries. This LA-mediated antiapoptotic effect was further markedly reduced two weeks after discontinuation of therapy.

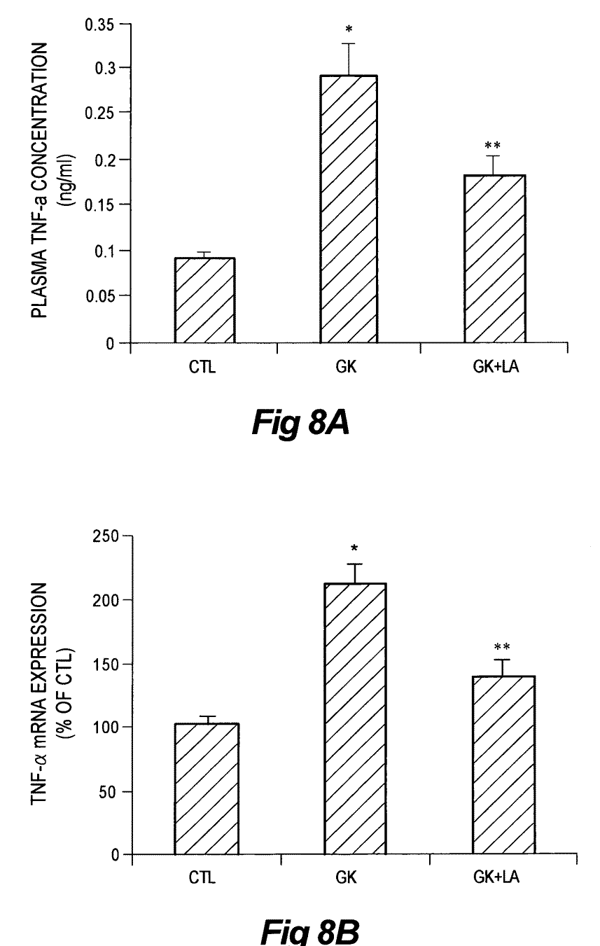

An elevation in NAD(P)H oxidase activity in connection with a high rate of apopototic cell death during diabetes may stem from vascular proinflammatory phenotype exemplified by enhanced activity of TNFα. Testing this possibility dictates the assessment of the status of TNF-α in diabetes. The results from these studies confirms that diabetes related upregulation in the rate of expression of TNF-α, both in terms of protein (plasma) and mRNA (aorta) levels, respectively illustrated in FIGS. 8A and 8B. Reversal of the above abnormalities was achieved by the institution of LA chronic therapy. In FIGS. 8A and 8B, levels of TNF-α were determined in plasma and aorta using, respectively, Eliza and QRT-PCR based techniques. Data are expressed as means±SEM of at least 7 animals/group.

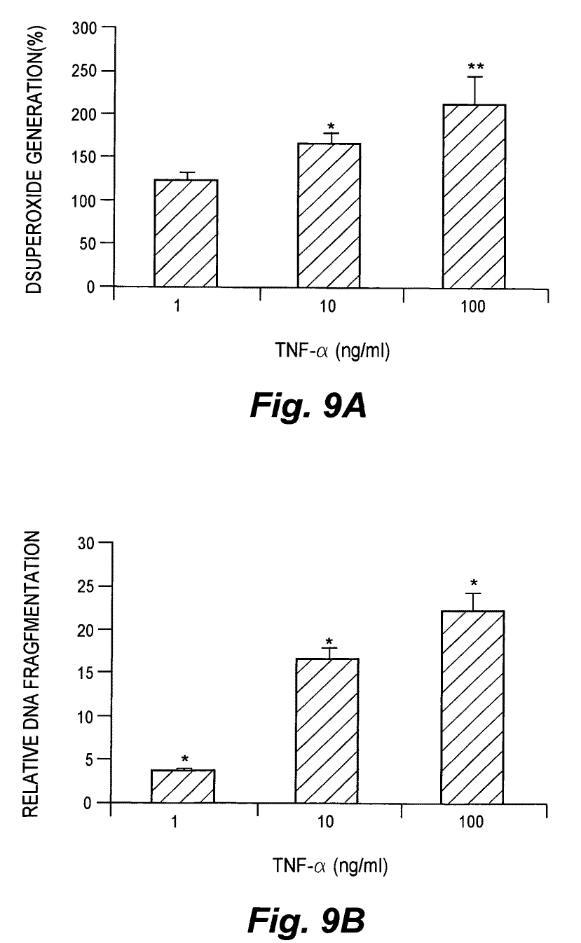

Further, the experiments have found that exogenous TNF-α administration mimics vascular diabetic phenotype. Cultured arteries derived from non-diabetic control animals were exposed in vivo to TNF-α and various other parameters, including: O₂— generation, Ach-induced relaxation, DNA fragmentation and caspase activity, which were all measured. As shown in FIGS. 9A and 9B, the data reveal that the rate of O₂— generation, caspase 3/7 activity and the levels of DNA fragmentation were elevated in response to TNF-α treatment. In contrast, this proinflammatory cytokine impaired Ach-induced vasorelaxation (shown in FIG. 9C). It should be noted that pretreatment with LA partially reversed the above TNF-α-induced abnormalities. FIGS. 9A, 9B and 9C illustrate concentration dependence of TNF-α vascular actions. Superoxide generation is shown in FIG. 9A, relative DNA fragmentation is shown in FIG. 9B, and acetylcholine induced vasorelaxation is shown in FIG. 9C. Data are expressed as means±SEM of at least 7 animals/group.

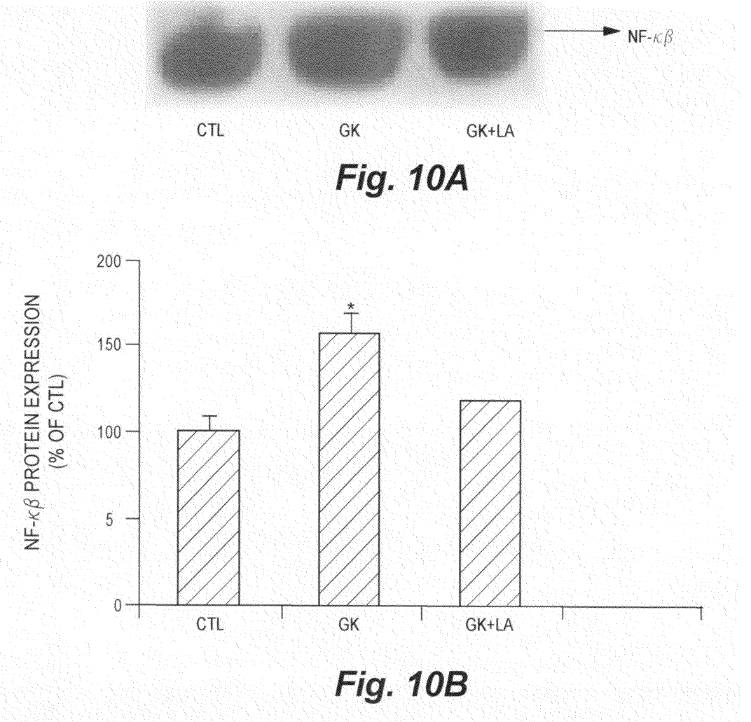

Further, the experiments revealed that LA mitigates diabetes-induced increases in vascular NF-κβ activity. It is well known that TNF-α enhances the activity of NF-κβ, most probably via H₂0₂— mediated mechanisms. Using data showing that both TNF-α and H₂0₂ levels were elevated in diabetic vascular tissues, NF-κβ activity was assessed using a western blotting-based technique with an antibody (anti P65) that specifically recognizes the active form of this transcription factor. The data reveals that NF-kβ level is high in vascular diabetic nuclei and this abnormality was reversed with LA chronic therapy, as shown in FIGS. 10A and 10B. FIGS. 10A and 10B illustrate aortic nuclear contents of immunoreactive NF-κβ in control (CTL), diabetic (GK) and LA treated diabetic rats (GK+LA). Nuclear localization of NF-κβ in aortic tissues was determined using differential centrifugation and western blotting-based techniques. FIG. 10A shows representative western blot analyses of Nf-κβ protein expression in aortic tissues of CTL, GK and GK+LA rats. FIG. 10B shows averaged densitometric data for GK and GK+LA groups expressed as a percentage of change over the CTL values expressed as 100%. Data are expressed as means±SEM of at least 7 animals/group.

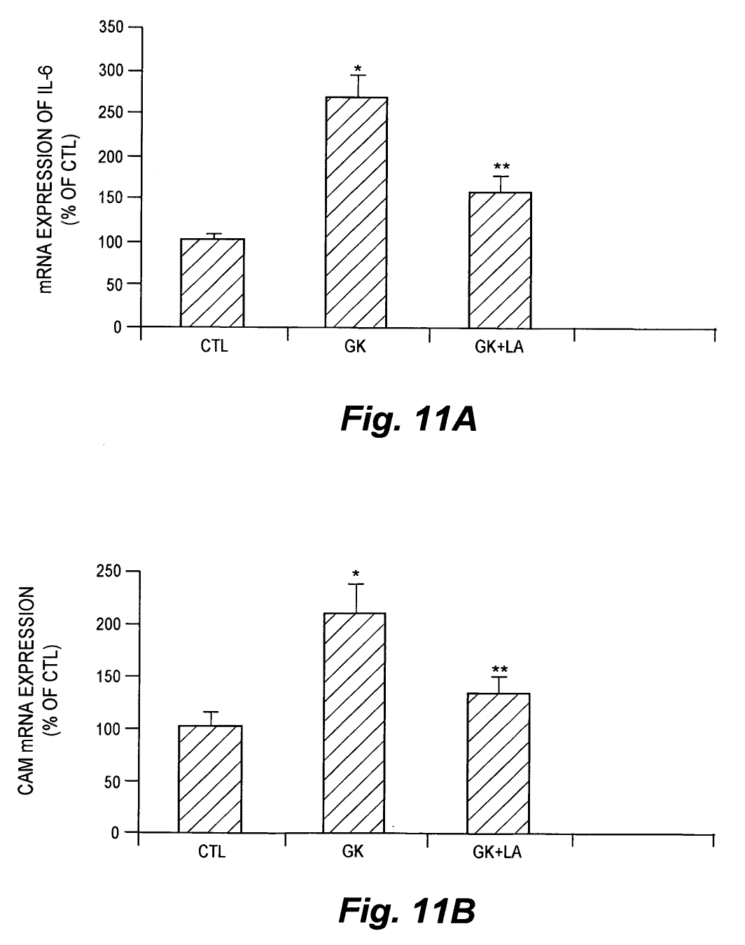

Further, the experiments revealed that LA counteracts diabetes-mediated upregulation of vascular proinflammatory markers. An expression of a number of inflammatory markers, including IL-6 and intracellular adhesion molecule (1 CAM-1), were measured in control, diabetic and LA-treated diabetic vessels. The results confirmed marked elevation in the vascular expression of both MCP-1 and CAM-1 during diabetes, as shown in FIGS. 11A and 11B. This diabetic vascular proinflammatory phenotype was partially reversed with LA therapy. FIGS. 11A and 11B show vascular expression of proinflammatory mediators in control (CTL), diabetic (GK) and LA-treated diabetic rats (GK+LA). Aortic expression of IL-6 is shown in FIG. 11A and aortic expression of intracellular adhesion molecule (ICAM) is shown in FIG. 11B. Both were determined using QRT-PCR based techniques. Data are expressed as means±SEM of at least 7 animals/group.

The above experiments have shown that LA prevents impairment of endothelial vasodilatation induced by oxidative stress in GK rats. Specifically, during diabetes, LA attenuates the ability of oxidative stress to decrease endothelial vasodilatation by interfering with signaling through the TNF-α/NF-κβ pathway, as shown in GK rats.

Diabetes is usually accompanied by an increased production of ROS and free radicals, or by impaired antioxidant defenses, which are widely accepted as important in the development and progression of diabetes complications. Oxidative stress also facilitates endothelial cell dysfunction. In this context, attenuated endothelium-dependent acetylcholine-induced relaxation has been reported in different vascular beds of human and animal models of diabetes. A number of cellular mechanisms have been suggested to account for impaired endothelium-dependent vasodilatation, including an actual synthesis/release of hydroxyl radicals. In the above experiments, a decline in Ach-induced relaxation of rat aorta was confirmed in GK diabetic rats, which appeared to be ameliorated with LA (as shown in FIG. 1). Overall, the development of endothelial dysfunction in aortic tissue of diabetic rats is most likely linked to an exaggerated production of O₂—. This enhancement in the production of O₂— may result in inactivation of NO and generation of peroxynitrite, as reflected by an increased aortic content of 3-nitrotyrosine.

The resulting decrease in NO availability might be involved in the impairment of NO dependent relaxation. Accordingly, oxidative degradation of NO caused by increased O₂— secondary to overactivity of NADH/NAD(P)H oxidase provides a reasonable explanation for the diminished response to Ach in the aorta of GK rats. It should be noted that the results do not exclude a role for other potential sources of O₂— (e.g., xanthine oxidase, mitochondrial flavoproteins) within diabetic vascular cells. Further, the observation that responses to sodium nitroprusside are altered in aortic tissue of GK rats suggests that other molecular mechanisms (e.g., diminished expression and activity of vascular smooth muscle cell guanylate cyclase) may also contribute to impaired vasodilatory responsiveness during diabetes. Both apocyanin and tiron improved Ach-induced relaxation in diabetic arteries, consistent with the concept that upregulation of NAD(P)H oxidase activity is responsible, at least in part, for diabetes-induced endothelial dysfunction (as seen in FIG. 1). The above findings are in accordance with prior results demonstrating diminution in Ach-based vascular relaxation in human and animal model of diabetes.

The underlying cellular and molecular mechanisms associated with diabetes-related endothelial dysfunction were explored in the context of a number of possibilities, including augmented production of O₂— and an imbalance in the rate of reactive oxygen/nitrogen species production and disposal within the microenvironment of the vessels. With regard to this connection, lucigenin chemiluminescence measurement revealed that the aorta of GK diabetic rats exhibited a marked increase in O₂— production, which was inhibited by apocynin and diphenyleneiodionium (as shown in FIG. 2). It should be noted that LA action on diabetic aortic O₂— generation mimics those produced by apocynin and diphenyleneiodonium.

Additionally, the results demonstrated that diabetic vessels exhibited a marked increase in the number of ethidium bromide (EB) positive nuclei, both in the endothelium and media, when compared to non-diabetic controls (as shown in FIG. 3). Nuclear EB fluorescence was significantly reduced in LA-treated diabetic rats. This phenomenon appears to be due to an effect of LA treatment in GK vascular tissues, compared with their corresponding Wistar control values. The level of this free radical was elevated in the aortic segment of the GK rats. Thus, the LA treatment in diabetic vessels represents a compensatory mechanism to counterbalance endothelial dysfunction induced by diabetes-dependent oxidative stress.

NADH/NAD(P)H oxidase, xanthine oxidase, a dysfunctional NO synthetase, or mitochondrial flavoproteins, represent an important source for ROS generation within vascular endothelial and smooth muscle cells. These ROS based enzymatic sources are subject to alterations by a variety of physiological and pathophysiological states, including diabetes. Further, mitochondrial flavoprotein-mediated increases in O₂— generation have also been observed in bovine aortic endothelial cells cultured under hyperglycemic conditions. The NAD(P)H oxidase system constitutes a pivotal signaling element in the genesis of endothelial dysfunction and is widely accepted to account for the majority of superoxide generation in the vascular endothelial and smooth muscle cells. Thus, the hypothesis that treatment with LA attenuated the stimulation of NADH/NAD(P)H oxidase and its contributions to a diabetes related increase in vascular O₂— production was examined. This proposition is supported by the above findings, which demonstrate that an enhancement in NAD(P)H oxidase driven O₂— generation in is exhibited in diabetic aorta, and which is significantly attenuated following the LA injection (as shown in FIG. 4). The increased lucigenin chemiluminescence of diabetic vessels may be substantially inhibited by diphenyleneiodonium and apocyanin.

The vascular NAD(P)H oxidase consists of at least 3-5 subunits, with the membrane-bound cytochrome b558, P22^{phox}, and gp91^{phox} being important for electron transport or the reduction of molecular oxygen to O₂—. Apocynin acts by interfering with the NAD(P)H subunit assembly in the membrane and is therefore a more specific inhibitor than diphenyleneiodonium. Experimentation using a western blotting-based technique and qRT-PCR revealed that the protein abundance of pg91phox and nox-1 subunits of NAD(P)H oxidase were reduced in aortic tissue of GK diabetic rats treated with LA (as shown in FIG. 5). In the above experiments, LA treatment also reduced the rate of gene expression of pg 9^{phox}, and nox-1 subunits.

Overall, the above data are consistent with the concept that the diabetic aorta exhibits a heightened state of oxidative stress. The consequences of this phenomenon upon biological molecules, including lipids and proteins, were determined. As the above results show, with specific reference to FIGS. 6A and 6B, the levels of both protein-bound carbonyls and the thiobarbituric acid reactive substances (an indicator of lipid peroxidation) were elevated in diabetic aorta by 45% and 60% respectively. LA treatment partially reversed the oxidative stress-mediated damage to the lipid and protein molecules during diabetes. Taken together, the inhibition of O₂— production by LA, in connection with the decreased expression of gp91^{phox} and nox-1 (shown in FIG. 5) in aortic tissue of GK rats are in accordance with the concept that the NAD(P)H oxidase in the diabetic state is hyperactive and that LA, via reducing its activity and expression, may contribute, at least in part, to the overproduction of O₂— in diabetic vessels.

Cytotoxic DNA fragmentation and caspase activities are sensitive indicators of endothelial cell death in blood vessels. Results from the above experiments revealed that the rate of DNA fragmentation in diabetic tissue was elevated by 75% over corresponding control values (as shown in FIGS. 7A and 7B). There was also an increase caspase 3/7 activity in diabetic vessels. Chronic LA treatment significantly reduced DNA fragmentation rate by 42% and caspase 3/7 by 48% in diabetic arteries. This LA-mediated antiapoptotic effect was markedly reduced two weeks after discontinuation of therapy.

Furthermore, in the above experiments, cultured arteries derived from non-diabetic control animals were exposed in vivo to TNF-α and various parameters, including O₂— generation, Ach-induced relaxation, DNA fragmentation and caspase activity, which were all measured. The data revealed that the rate of O₂— generation, caspase 3/7 activity and the levels of DNA fragmentation were elevated in response to TNF-α treatment (as shown in FIGS. 9A, 9B and 9C). In contrast, this proinflammatory cytokine impaired Ach-induced vasorelaxation. Additionally, pre-treatment with LA partially reversed the above TNFα-induced abnormalities. It is well established that TNFα enhances the activity of NF-kβ probably via H₂O₂— mediated mechanisms. The experimental data revealed that the NF-κβ level is high in vascular diabetic nuclei, and that this abnormality was reversed with LA chronic therapy, as shown in FIGS. 10A and 10B.

Factors affecting the expression of endothelial adhesion molecules, therefore, are important in regulating vascular inflammatory processes. Activation of the transcription factor NF-κβ; e.g., by inflammatory cytokines, is required for the transcriptional activation of endothelial cell adhesion molecules. In the above experiments, it was found that LA inhibits NF-κβ activation and adhesion molecule expression in aortic tissue of GK rats. The data demonstrate that LA effectively inhibits TNF-α-stimulated mRNA and TNF-α plasma concentration (shown in FIGS. 8A and 8B) and consequent attenuated endothelial vasodilatation (shown in FIGS. 9A, 9B and 9C), as well as LA inhibiting NF-κβ protein expression (shown in FIGS. 10A and 10B).

In the above experiments, an expression of a number of inflammatory markers, including IL-6 and intracellular adhesion molecule (1CAM-1), were measured in control, diabetic and LA-treated diabetic vessels. The results confirmed marked elevation in the vascular expression of both MCP-1 and CAM-1 during diabetes (as shown in FIGS. 11A and 11B). This diabetic vascular proinflammatory phenotype was partially reversed with LA therapy. The data that LA inhibits mRNA expression for ICAM-1 and IL-6 indicates that LA inhibits binding of NF-κβ to the upstream regulatory promoter sequences of these genes. The data strongly suggest that LA inhibits TNF-α-induced endothelial activation by affecting the NF-kβ/Ikβ signaling pathway at the level (or upstream) of IKK, rather than by preventing DNA binding of NF-kβ.

This conclusion is further supported by observations that LA also inhibits diabetes-induced adhesion molecule expression in aortas of GK rats and NF-κβ activation in other cells. NF-κβ has been proposed to be a redox-sensitive transcription factor. In most cell types, NF-κβ can be activated by a diverse range of stimuli, suggesting that several signaling pathways are involved.

The observed anti-inflammatory action of LA in aortic tissue of GK rats extends to many other important mediators of inflammation, in a variety of cells and tissues. It is believed that LA exerts vasculoprotective effects, via mechanisms involving the downregulation of the TNFα/NFκβ signaling pathway.

It is to be understood that the present invention is not limited to the embodiment described above, but encompasses any and all embodiments within the scope of the following claims.

We claim:

1. A method of treating diabetes-related vascular complications, comprising the step of administering to a patient a therapeutically effective dosage of alpha-lipoic acid or pharmaceutically acceptable salts thereof for the treatment of diabetes-related vascular complications.

2. The method of treating diabetes-related vascular complications as recited in claim 1, wherein the step of administering to the patient the therapeutically effective dosage of alpha-lipoic acid includes delivery of the alpha-lipoic acid to the patient through oral delivery.

3. The method of treating diabetes-related vascular complications as recited in claim 2, wherein the alpha-lipoic acid is delivered to the patient in a dosage of between approximately 100 mg. and 300 mg.
